(19) United States
(12) Patent Application Publication
(10) Pub. No.: US 2024/0096504 A1
(43) Pub. Date: Mar. 21, 2024

(54) DRUG CONTAINER LABELING APPARATUS AND METHOD FOR DISTINGUISHING BETWEEN A PLURALITY OF DRUG CONTAINERS

(71) Applicant: Codonics, Inc., Middleburg Heights, OH (US)

(72) Inventors: Gary Keefe, Brecksville, OH (US); Lawrence Srnka, Northfield Center, OH (US)

(21) Appl. No.: 18/238,942

(22) Filed: Aug. 28, 2023

Related U.S. Application Data
(60) Provisional application No. 63/401,338, filed on Aug. 26, 2022.

(51) Int. Cl. G16H 70/40; G06K 7/10; G06K 19/077; G06V 30/14; G06V 30/224

(52) U.S. Cl. CPC G16H 70/40; G06K 7/10316; G06K 19/07758; G06V 30/1448; G06V 30/2247

(57) ABSTRACT

Provided is a drug labeling apparatus and method that involves scanning a barcode provided to a first drug vial to obtain information related to a first drug stored by the first drug vial. Data stored by a computer-readable medium of a first RFID tag associated with the first drug vial is read. Data stored by a computer-readable medium of a second RFID tag associated with a second drug vial is also read. The data determined based on the barcode is compared to the data read from the media provided to the first and second RFID tags. It is determined that the data read from the medium provided to the first RFID tag corresponds to the first drug based on the comparison result. Label content including information derived from the barcode and information derived from the data read from the medium provided to the first RFID tag is generated.

### BACKGROUND OF THE INVENTION

### 1. Field of the Invention

This application relates generally to a method and apparatus for preparing a label for a drug delivery container and, more specifically, to a method and apparatus for distinguishing between a plurality of drug source containers present at a location where the drug delivery container is being prepared.

### 2. Description of Related Art

Industry and/or government regulations require delivery containers used to administer drugs to patients to be labeled with information that identifies those drugs. To comply with regulations and industry standards, labels on drug delivery containers must include certain information about the drug, such as drug name for example, and other information pertaining to the drug. In addition to human-readable content, labels for drug delivery containers have evolved to also include computer-readable codes such as barcodes, RFID tags, or other information about the drug to be administered.

Conventional labeling systems have relied on the manual entry of information to be included on labels, in human-readable and/or computer-readable form, creating an opportunity for human error to be introduced into the labeling process. Other conventional labeling systems have scanned a barcode encoding the National Drug Code (“NDC”) number for a drug. The NDC number would then be used to lookup data for the drug in a formulary to extract the drug name to be included on the label being generated. However, the information that is encoded by the NDC represented by barcodes is limited, and specific to the process of identifying a drug for administration to a patient.

Other computer-readable codes are being explored for storing additional information in an effort to expand the usefulness of information encoded on labels provided to drug source vials. For example, radio frequency identification tags (“RFID”) tags store information in a non-transitory computer-readable medium. This information can be read by interrogating the RFID tag when the RFID tag is placed within an electromagnetic field generated by a RFID reader. However, the electromagnetic field may reach a plurality of RFID tags provided to different drug vials, interrogating and reading the plurality of RFID tags within the electromagnetic field. Accordingly, a technical problem associated with using RFID tags to label drug vials, for example, is that a RFID reader will read multiple RFID tags present at the point of administration, instead of only the RFID tag provided to the drug vial of interest.

### BRIEF SUMMARY OF THE INVENTION

According to one aspect, the subject application involves a drug labelling apparatus including an optical code reader. The optical code reader includes an optical imager that captures pattern data indicative of a pattern of symbols included in a first computer-readable code provided to a first drug vial. The pattern data represents information related to a first drug stored by the first drug vial. A RF code reader provided to the drug labelling apparatus includes an antenna, and a signal generator that generates an interrogation signal to be transmitted by the antenna to: (i) generate a field that encompasses a first RFID tag associated with the first drug vial and a second RFID tag associated with a second drug vial, and (ii) read data stored by non-transitory computer-readable media provided to the first RFID tag and the second RFID tag. A differentiator module of the drug labelling apparatus compares: (i) a portion of the information about the first drug determined based on the first computer-readable code, and (ii) a portion of the data read from the non-transitory computer-readable media provided to the first RFID tag and the second RFID tag to generate a comparison result. The differentiator module determines that the data read from the non-transitory computer-readable medium provided to the first RFID tag corresponds to the first drug stored by the first drug vial based on the comparison result. A content generation module of the drug labelling apparatus includes a computer processing unit programmed with computer-executable instructions to generate label content comprising information derived from the pattern data and information derived from the data read from the non-transitory computer-readable media provided to the first RFID tag. A publishing module of the drug labelling apparatus generates a drug label that is to be applied to a drug delivery container by applying the label content to an adhesive-backed label stock.

According to some aspects, the optical code reader includes a barcode reader that reads the pattern data from a one-dimensional and/or two-dimensional barcode. The barcode reader extracts a standardized code, such as a National Drug Code (“NDC”) for example, that is compliant with a standard approved by a regulatory body, such as the Food and Drug Administration for example, comprising a numeric, alphabetic, or alpha-numeric string from the pattern data to identify at least a name of the first drug. The NDC includes a string of numbers arranged in three segments, and the name of the first drug is encoded by one of the three segments.

According to some embodiments, the RF code reader reads at least supplemental information, that is different from the information related to the first drug represented by the pattern data, from the non-transitory computer-readable medium provided to the first RFID tag. For example, the supplemental information can include at least one of: an expiration date of the first drug stored by the first drug vial, and a lot number assigned to the first drug stored by the first drug vial by a supplier of the first drug vial. According to some embodiments, the supplemental information is absent from the information related to the first drug encoded by the pattern data. Thus, the drug labeling apparatus can supplement information related to the first drug encoded by the pattern data. For example, the pattern data can be interpreted to identify the drug name, and the supplemental information can be interpreted to obtain the expiration date and/or time, for example. A label can be printed with the combination of the drug name, expiration date and/or time in human-readable form, computer-readable form, or in both human-readable form and computer-readable form.

According to some embodiments, the drug labeling apparatus includes a source module that identifies a source of the data read from the non-transitory computer-readable medium provided to the first RFID tag, designates the source of the data to be a reliable source or an unreliable source and, in response to determining that the source of the data is the unreliable source, alerts a user of the drug labeling apparatus to a potential property of the drug.

According to some embodiments, the source module can identify the source as the reliable source or the unreliable source based on a source identifier included in the data read from the non-transitory computer-readable medium provided to the first RFID tag. For example, the source module can identify the source as a reliable source in response to determining that the data read from the non-transitory computer-readable medium provided to the first RFID tag comprises substantive information about the first drug. Examples of the substantive information about the first drug include at least one of: a name of the first drug, an expiration date of the first drug, and a lot number of the first drug.

According to some embodiments, the source module can identify the source as the unreliable source in response to determining that the data read from the non-transitory computer-readable medium provided to the first RFID tag lacks substantive information about the first drug, instead storing a reference number such as a serial number. The reference number can be used to locate a storage location in a database where information about the first drug can be retrieved, instead of being read directly from the RFID tag. The data read from the non-transitory computer-readable medium provided to the first RFID tag can optionally be determined to lack the substantive information about the first drug if the data read from the non-transitory computer-readable medium provided to the first RFID tag: (i) does not comprise a name of the first drug, an expiration date of the first drug, or a lot number of the first drug, and (ii) comprises a reference number that is used to lookup drug-specific information from a database.

According to some embodiments, the source module can interfere with operation of the publishing module to generate the drug label, and requires user confirmation of at least a portion of the label content before causing the publishing module to generate the drug label.

According to some embodiments, the subject application involves a method of producing a label for a drug delivery container. A barcode provided to a first drug vial is scanned with an optical code reader using an optical imager, to obtain information related to a first drug stored by the first drug vial. Data stored by a non-transitory computer-readable medium provided to a first RFID tag associated with the first drug vial, and data stored by a non-transitory computer-readable medium provided to a second RFID tag associated with a second drug vial is read with a RF code reader. A differentiator module compares: (i) a portion of the information about the first drug determined based on the barcode, and (ii) a portion of the data read from the non-transitory computer-readable media provided to the first RFID tag and the second RFID tag to generate a comparison result. The differentiator module determines that the data read from the non-transitory computer-readable medium provided to the first RFID tag corresponds to the first drug stored by the first drug vial based on the comparison result. A content generation module generates label content comprising information derived from the barcode and information derived from the data read from the non-transitory computer-readable medium provided to the first RFID tag. A publishing module produces a drug label that is to be applied to a drug delivery container for administering the first drug by applying the label content to an adhesive-backed label stock.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows illustrative embodiments of drug vials containing different drugs, the drug vials comprising a label including a barcode and a RFID tag storing data associated with the respective drugs;

FIG. 2 shows an illustrative embodiment of a RFID tag;

FIG. 3 shows an illustrative embodiment of a drug labeling apparatus scanning a barcode applied to a first drug vial held by a user, wherein the user is holding the first drug vial between two fingers and a second drug vial in a palm;

FIG. 4 is a block diagram schematically depicting components of a drug labeling apparatus for producing labels to be applied to medicinal substances in a medical facility; and

FIG. 5 is a flow diagram schematically depicting an embodiment of a method of producing a label for a drug delivery container.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

The present disclosure describes a labeling apparatus and method that uses a first computer-readable code to distinguish between a plurality of vials supporting a second computer-readable code. Examples of the first computer-readable code include a barcode or other such code that is optically read through line-of-sight interrogation, or is placed within close proximity of the compatible scanner. Due to the limited range or line-of-sight requirement of the compatible scanner, a drug labeling apparatus can read the first computer-readable code without also reading another instance of the computer-readable code that also requires line-of-sight interrogation as part of the same scanning operation. In other words, a user can hold a plurality of drug vials labeled with barcodes in the same hand, and scan each barcode individually, one at a time, without also scanning a nearby barcode.

The first computer-readable code, such as a barcode, encodes a limited amount of information, and is applied to the vial by a source (e.g., manufacturer, compounder, etc.) of the drug. For example, the barcode can encode a standard-compliant reference number such as a National Drug Code (“NDC”), established by the Food and Drug Administration. The NDC can be used to lookup information about the drug from a formulary or other database. However, because the NDC is used to label all vials containing a specific type of drug from a particular manufacturer, and does not uniquely identify one vial containing a drug from a manufacturer from another vial containing that same drug from the same manufacturer. Thus, the NDC does not contain certain information, such as expiration date or lot number for example, that is specific to individual drug vials containing the same drug from the same manufacturer.

A second computer-readable code such as a radio frequency identification (“RFID”) tag can be applied to drug vials, and store vial-specific information in a computer-readable medium included as part of the RFID tag. The vial-specific information such as the expiration date and/or lot number, for example, can be stored in the computer-readable medium of the RFID tag during a manufacturing process. However, RFID tags are read when placed within a field generated by a RF code reader. A plurality of RFID tags can be placed within the field generated as part of the same RFID interrogation process. If multiple RFID tags are read as part of the same RFID interrogation process, traditional RF code readers are unable to determine which RFID tag a user intended to interrogate and read data from the respective computer-readable medium.

To address at least some of the problems associated with interrogating a plurality of RFID tags within a common field, some embodiments of the subject drug labeling apparatus compare information obtained as a result of scanning a barcode provided to one of the drug vials, to information obtained as a result of interrogating the RFID tags provided to the plurality of drug vials. For example, if the plurality of drug vials contain different drugs, the drug vials will be labeled with different barcodes, encoding different reference numbers such as the NDC. Interrogating a plurality of RFID tags provided to the different drug vials in the field will return a plurality of results. The information for reach result can optionally include the NDC, drug name, or other information that can be compared to a portion of the information obtained by scanning the barcode on at least one of the drug vials. A match between information obtained by scanning the barcode and information read from the RFID tag provided to one of the vials can be used to identify the drug vial of interest, from among the plurality of drug vials with RFID tags in the interrogation field. Accordingly, the subject drug labeling apparatus can distinguish between a plurality of drug vials provided with RFID tags within an interrogation field.

A portion of the information read from the RFID tag provided to the drug vial of interest can be used to supplement a portion of the information obtained by scanning the barcode provided to the drug vial of interest. The combined information can be used to generate label content in human-readable form, computer-readable form, or both human-readable form and computer-readable form. The drug labeling apparatus prints the label content onto adhesive-backed label stock to produce a drug label to be applied to a drug delivery container such as a syringe or IV bag, for example.

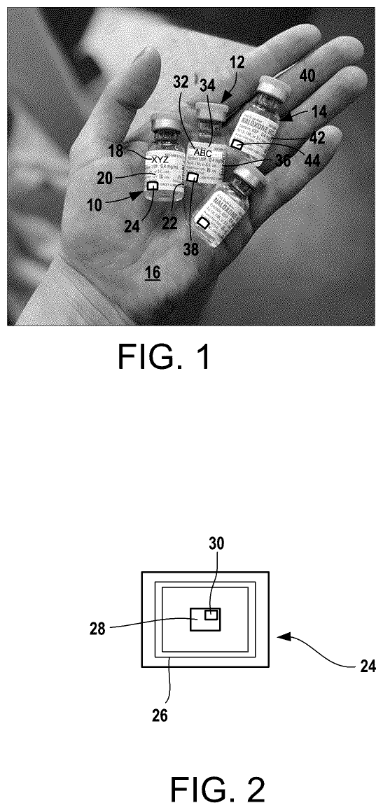

With reference to the drawings, FIG. 1 shows a plurality of drug vials 10, 12, 14. The drug vials 10, 12, 14 are often small enough to fit in the palm of a user's hand 16, and contain different drugs. For example, drug vial 10 contains drug XYZ, as identified by human-readable printed characters 18 on a label 20 applied to the drug vial 10. The label 20 also includes a first computer-readable code, such as a barcode 22 for example, that is optically read by an optical code reader (e.g., barcode scanner) with an optical imager (e.g., CMOS, CCD or other sensor circuit). For the sake of brevity and clarity the first computer-readable code is described herein as a one-dimensional barcode 22, however, the present disclosure is not so limited. The barcode 22 encodes information associated with the drug stored in the drug vial 10, and can be generic to the drug stored in the packaging (e.g., vial size) produced by the manufacturer, preparer, compounder or packager that is the source of the drug vial 10 for medical facilities. For example, the barcode 22 can encode the NDC of the drug contained in the drug vial 10. The NDC includes a string of characters divided into a plurality (e.g., three) segments, that identify the labeler, product, and trade package size. Thus, multiple units of the drug vial 10 containing the drug XYZ, supplied by the same source in the same trade package size, will be labeled by the same NDC. Although the NDC is a standardized number established by the Food and Drug Administration to identify the labeler, product, and trade package size, the NDC does not encode information that identifies information specific to separate units of the drug vial 10. For example, the expiration date and/or time, and/or the lot number of individual units of the drug vial 10 are not read directly from the barcode 22.

Because line-of-sight is required between the barcode 22 and a barcode scanner to scan the barcode 22 as described herein, a single barcode 22 can be read during each label-reading operation by exposing the single barcode 22 to the barcode scanner at a time. According to some embodiments, the reading zone of the barcode scanner can optionally be limited to read a single barcode 22 at a time. Thus, the potential to inadvertently read a plurality of barcodes concurrently can be avoided.

The drug vial 10 can also be labeled with a second computer readable code, such as a RFID tag 24 for example, that electronically stores information about the drug contained by the drug vial 10. An illustrative example of the RFID tag 24, shown schematically in FIG. 2, includes an antenna 26 electrically connected to a simple processing unit 28 including a non-transitory, computer-readable memory 30. The drug information to be read from the RFID tag 24 is stored in the memory 30 of the processing unit 28. The processing unit 28 is said to be “simple” in that it lacks a power source onboard the RFID tag 24. Instead, the RFID tag 24 is passive. An electric current is induced within the antenna 26 to electrically excite the RFID tag 24 in response to being exposed to, or otherwise interrogated by a field generated by a RFID reader as described herein. When excited, the processing unit 28 of the RFID tag 24 generates a data signal comprising drug data, which is transmitted by the antenna 26 back to the RFID reader. Examples of the drug data transmitted as part of the data signal includes at least one of: a drug name, a drug concentration, a standardized or other reference number assigned to the drug, an expiration date and/or time of the drug, expiration information indicative of when a syringe or other delivery container expires after being prepared from the drug vial 10, a drug class, a color code associated with a drug class, a lot number identifying a date and/or time of manufacture, a serial number, any other drug-related information, and the like.

The RFID tag 24, if applied to the drug vial 10 by a manufacturer, compounder or packager of the drug that is identified by the corresponding segment of the NDC, stores drug data in the memory 30 that can be relied on as a source of truth. In other words, the drug data itself can be retrieved directly from the memory 30, as opposed to being looked up and retrieved from a formulary 84 (FIG. 4) external of the memory 30, the RFID tag 24 and the drug data in the memory 30 are deemed to be reliable. In contrast, if the RFID tag 24 applied to the drug vial 10 stores a reference number such as a serial number, which is to be used to lookup drug data from a formulary 84 (FIG. 4) or other source external of the memory 30, the RFID tag 24 is deemed to be unreliable, and determined to have been applied to the drug vial 10, or the data stored therein by a third party.

Referring once again to FIG. 1, a second drug vial 12 contains drug ABC, as identified by human-readable printed characters 32 on a label 34 applied to the second drug vial 12. Similar to the drug vial 10, the second drug vial 12 includes a first computer-readable code, such as a barcode 36 for example, and a second computer-readable code, such as a RFID tag 38 for example. The barcode 36 and the RFID tag 38 are similar to the barcode 22 and the RFID tag 24 described above for the drug vial 10, respectively, but encode and/or store drug information specific to the second drug vial 12 and/or the drug stored in the second drug vial 12. Accordingly, a detailed description of the barcode 36 and the RFID tag 38 is omitted at this point. Further, a third drug vial 14 can also include a label 40 that includes a barcode 42 and/or a RFID tag 44, which can be similar to the barcode 22 and the RFID tag 24 described above for the drug vial 10, respectively, but encode and/or store drug information specific to the third drug vial 14 and/or the drug stored in the third drug vial 14.

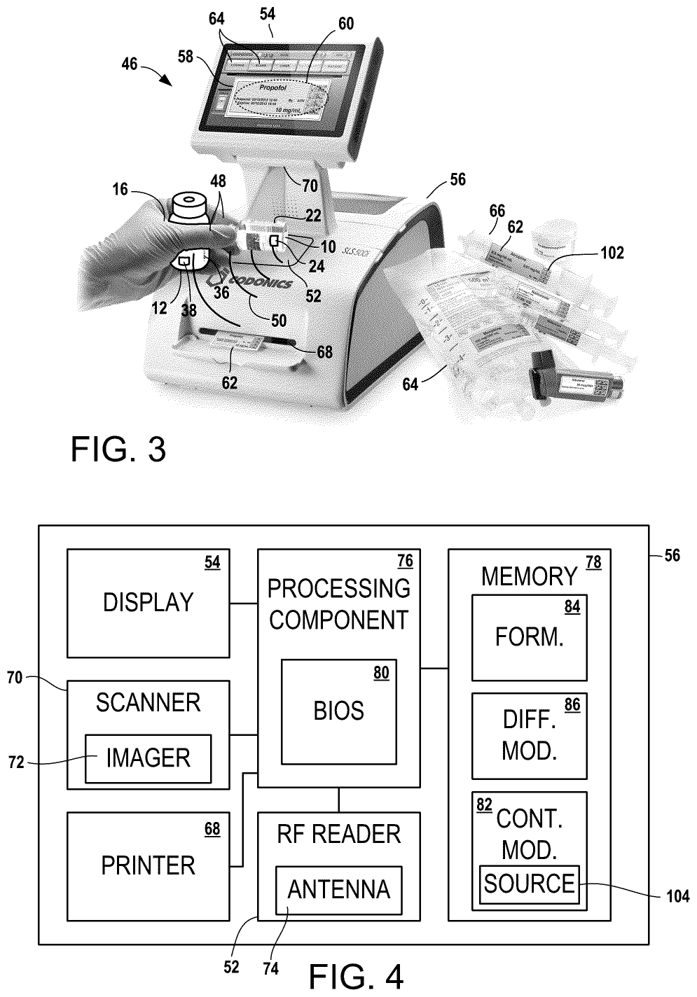

FIG. 3 shows an illustrative embodiment of a drug labeling apparatus 46 scanning a barcode 22 applied to the first drug vial 10 held by a user. The user is holding the first drug vial 10 between two fingers 48, and the second drug vial 12 in a palm 16 of the user's hand. When preparing a plurality of drug delivery containers to be used for administering different drugs, such handling of the drug vials 10, 12 is believed to be common to efficiently prepare the delivery containers. However, such handling procedures have interfered with the adoption of RFID tags 24, 38 on vials 10, 12 out of concern that a field 50 generated by a RF code reader 52 could concurrently interrogate the RFID tags 24, 38 provided to both vials 10, 12.

As shown in FIG. 3, the drug labeling apparatus 46 includes a touch-screen display 54 that can be pivotally coupled to a cabinet 56 to display a virtual label 58 to preview label content 60 that will be printed onto an adhesive-backed label stock to produce a drug label 62. The virtual label 58 is a computer-generated rendering of the drug label 62 that offers the user visual confirmation of the appearance of the physical drug label 62 to be printed by a printer 68. The display 54 can display soft keys 64 that, when touched by a technician or any other user, inputs data and commands into the drug labeling apparatus 46. The drug label 62 will be applied to a delivery container 66 (e.g., a syringe, IV bag, pill cup, etc.) to be used for administering a drug to a patient.

According to some embodiments, an optical code reader such as a barcode scanner 70, for example, is provided to the drug labeling apparatus 46 at a location where the drug vials 10, 12 can be held to expose the respective barcodes 22, 36 to an optical imager 72 (FIG. 4). The optical imager 72 includes a sensor and circuitry that captures an image of a pattern forming the barcodes 22, 36, and converts the image into a data structure that is processed to decode the information encoded by the barcodes 22, 36. For the embodiment shown in FIG. 3, the barcode scanner 70 is integrally formed in a bottom portion of the display 54 to read the barcode 22 applied to the drug vial 10 containing a drug supported beneath the display 54.

The drug labeling apparatus 46 can also include the RF code reader 52 arranged to interrogate the RFID tags 24, 38 when the respective drug vials 10, 12 are supported where the barcodes 22, 36 are read by the barcode scanner 70. Although the illustrative embodiments are described herein as including the RF code reader 52, other embodiments can include any computer-readable code reader that generates a field that can interrogate more than one computer-readable code present within the field. According to some embodiments, the RF code reader 52 includes an antenna 74 (FIG. 4) that conducts an electric current to generate a field 50. The field 50 induces an electric current in the antenna 26 of the RFID tag 24, which energizes the processing unit 28 of the RFID tag 24, causing the processing unit 28 to retrieve the respective drug data from the memory 30 of the RFID tag 24, and transmit the retrieved drug data via the antenna 26 back to the antenna 74 of the RF code reader 52. Similarly, with the second drug vial 12 being held in the palm 16 of the user, the RFID tag 38 is exposed to the field 50 that also interrogates the RFID tag 24 of the first drug vial 10. As a result, the drug labeling apparatus 46 reads two sets of drug data, one from each RFID tag 24, 38.

The drug labeling apparatus 46 also includes a cabinet 56 that houses or supports components that are operable to produce the drug label 62 as described herein. Illustrative embodiments of components that can be coupled to, or housed within the cabinet 56 are schematically illustrated by the block diagram of FIG. 4. The components can be formed from an arrangement of computer hardware such as ASICs, computer processors, programmable logic controllers and other circuitry; or a combination of computer hardware and computer-executable instructions. For example, a processing component 76, such as a computer processor for example, includes circuitry that executes computer-executable instructions stored in a non-transitory, computer-readable memory 78 such as a hard disk drive, read-only memory (“ROM”), random access memory (“RAM”), optical disc, or any other suitable memory device, or any combination thereof. The computer-executed instructions, when executed by the processing component 76, result in the performance of the method of generating a label for a medicinal substance described in detail below. A BIOS 80 is provided to load the operating system and other such administrative instructions stored in the memory 78 and manage hardware interface permissions of the drug labeling apparatus 46. The memory 78 of the drug labeling apparatus 46 can also store a drug formulary 84, which includes a database of drugs related to corresponding drug information such as a reference number (e.g., NDC), for example, that can be used to lookup drugs in the formulary 84.

The formulary 84 stored by the memory 78 is updatable, and contains a database of medicinal substances that can be identified by the drug labeling apparatus 46 and select information for each medicinal-substance entry in the database. The formulary 84 can optionally be stored, updated and deleted from the memory 78 by the introduction of a so-called smart drive comprising a USB compatible flash memory to the drug labeling apparatus 46. When the smart drive is introduced to the drug labeling apparatus 46, it establishes the formulary 84 of the drug labeling apparatus 46. Illustrative examples of the select information that can be provided for the medicinal-substance entries includes, but is not limited to, an ID number such as a NDC code, UPC code, EAN code, or any other identifying data that can be used to relate a barcode or other computer-readable code to the medicinal-substance entries; a sound file that, when played, audibly announces the name of the medicinal substance identified in response to scanning a machine readable code; warning data; or any combination thereof.

The memory 78 can optionally store computer-executable instructions that, when executed by the processing component 76, constitutes a differentiator module 86. Although the differentiator module is shown in FIG. 4 as computer-executable instructions that are used to program the processing component 76, alternate embodiments can include an application-specific integrated circuit, or any other configuration including circuitry, software, or a combination thereof.

Using the first drug vial 10 as an example, the differentiator module 86 compares a portion of information about the first drug obtained as a result of optically interrogating, or scanning, the barcode 22, to a portion of the information about the first drug obtained as a result of reading the memory 30 of the RFID tag 24. The differentiator module 86 generates a comparison result to confirm that the information read from the memory 30 corresponds to the first drug associated with the barcode 22. The information about the first drug obtained as a result of optically interrogating, or scanning, the barcode 22 can be compared to the information/data read from the memory 30 of each RFID tag 10, 12 within the field 50. According to some embodiments, the comparison of the information about the first drug obtained as a result of optically interrogating, or scanning, the barcode 22 can be compared to the information obtained from the memory 30 of RFID tags 24, 38 within the field 50 until a match is found, at which point the comparisons can end. Accordingly, if more than one RFID tag 24, 38 is within the field 50 and interrogated by the RF code reader 52, the processing component can compare information obtained in response to scanning the barcode 22 to the information obtained in response to reading the RFID tags 24, 38, to determine that the information from the RFID tag 24 corresponds to the information obtained by scanning the barcode 22. The barcode 22 and the RFID tag 24 are both applied to the first drug vial 10. The RFID tag 38 is coupled to the second drug vial 12, which contains a different drug than the first drug vial 10, so the information stored by the RFID tag 38 corresponds to a different drug than the information stored by the RFID tag 24. The differentiator module 86 can control generation of the label content 60 to exclude drug information specific to the different drug in the second drug vial 12 from being included in the label content 60 to be applied to the drug label 62 for the delivery container 66 that is to be prepared from the first drug vial 10.

Based on the comparison result, a content generation module 82 generates the label content 60 for the drug label 62. Although the content generation module is shown in FIG. 4 as computer-executable instructions stored in the memory 78 that are used to program the processing component 76, alternate embodiments can include an application-specific integrated circuit, or any other configuration including circuitry, software, or a combination thereof.

According to some embodiments, the label content 60 includes a combination of at least a portion of the information derived from the pattern data obtained by scanning the barcode 22, and a portion of the information obtained as a result of reading the RFID tag 24. According to alternate embodiments, the label content 60 can include information obtained as a result of reading the RFID tag 24, without including information obtained as a result of scanning the barcode 22, but the information obtained by scanning the barcode 22 can be used simply to select the RFID tag 24, from among the plurality of RFID tags 24, 38 (according to the embodiment in FIG. 3) present within the field 50.

In use, the drug labeling apparatus 46 determines drug information for a first drug from a plurality of computer-readable sources to prepare a drug label 62 to be applied to a drug delivery container 66. As part of the process, the drug labeling apparatus 46 can use information obtained from the plurality of computer-readable sources to exclude from the drug label 62, drug information obtained for a second drug from another computer-readable source interrogated during a read operation.

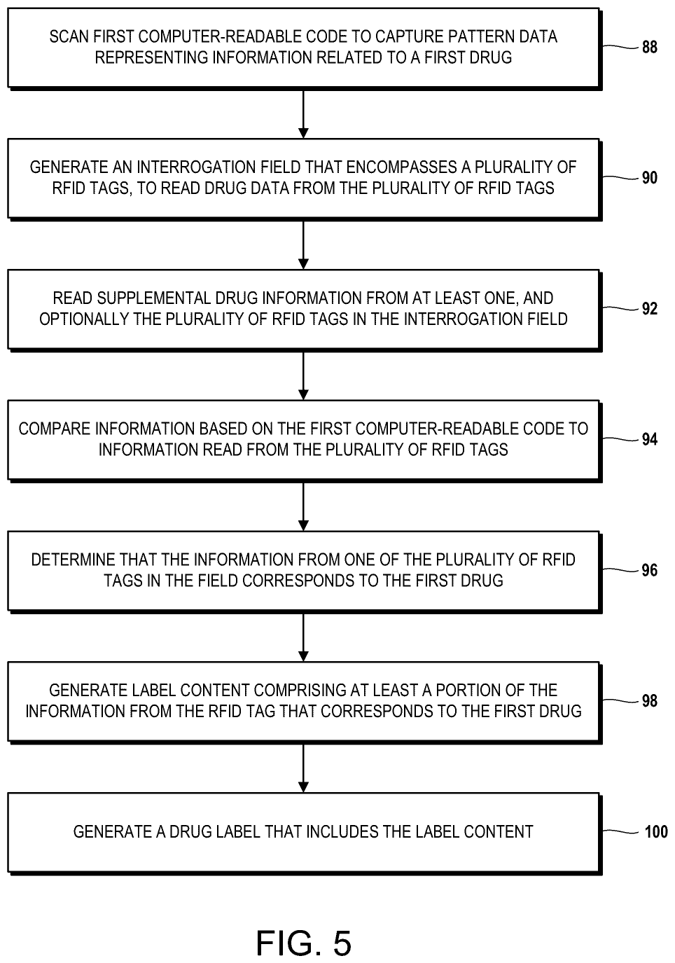

FIG. 5 shows a flow diagram graphically depicting an embodiment of the present method. To commence label preparation, a first drug vial 10 is held adjacent to the barcode scanner 70, to expose the barcode 22 applied to the label 20 of the first drug vial 10 to the optical imager 72. At block 88, the optical imager scans the barcode 22, which can be a one-dimensional or two-dimensional barcode for example, to obtain information related to a first drug contained in the first drug vial 10. For example, the barcode scanner 70 captures pattern data indicative of a pattern of symbols included in the barcode 22. The pattern data represents information such as a standardized code that is compliant with a standard approved by a regulatory body comprising a numeric, alphabetic, or alpha-numeric string from the pattern data related to the first drug stored in the first drug vial 10. The pattern data can be used by the barcode reader or transmitted to the processing component 76, which can decode the pattern data and/or use the pattern data to extract information about the first drug from the formulary 84. For example, the pattern data can encode the NDC or other reference number for the first drug, and the NDC can be determined by scanning the barcode 22 on the first drug vial 10. The NDC can be used to query a database included in the formulary 84, to identify a matching entry that includes, in association with the NDC, at least one of: a manufacturer, re-packager distributor or other source of the first drug; a drug name; specific strength, dose and formulation; and commercial package size, for example.

At block 90, the RF code reader 52 transmits an interrogation signal through the antenna 74 that generates the field 50 encompassing the first RFID tag 24 associated with the first drug vial 10 and the second RFID tag 38 associated with the second drug vial 12, to read information about the drugs in the drug vials 10, 12. The field 50 induces an electric current through the antenna 26 of each RFID tag 24, 38, causing the RFID tags 24, 38 to retrieve data from their respective memories 30, and transmit the retrieved data, which is received by the RF code reader 52 via the antenna 74.

At least a portion of the data received from the RFID tags 24, 38 falls within a category that corresponds to a category of information that is obtained as a result of scanning the barcode 22. For instance, at least a drug name can be determined by scanning the barcode 22, and at least the drug name can be received from the RFID tags 24, 38. As another example, at least a drug name can be determined by scanning the barcode 22, and at least the drug name can be received from the RFID tags 24, 38. As another example, at least the NDC can be determined by scanning the barcode 22, and at least the NDC can be received from the RFID tags 24, 38. As long as there is an overlap in the category of information that is determined based on the barcode 22 and the category of information that is read from the RFID tags 24, 38, a comparison can be conducted to determine whether the RFID tag 24 and/or the RFID tag 38 contains drug information corresponding to the drug contained in the first drug vial 10.

At block 92, the RF code reader 52 reads at least supplemental information from the RFID tag 24 and/or the RFID tag 38, that is different from the information related to the first drug that was obtained as a result of scanning the barcode 22. The supplemental information can include at least one of: an expiration date of the first drug stored by the first drug vial 10, and a lot number assigned to the first drug stored by the first drug vial 10 by a source of the first drug vial 10. Because the supplemental information is specific to individual drug vials, as opposed to all drug vials that have a common: source; drug name; and specific strength, dose and formulation; and commercial package size (collectively “standardized data”) for example, different vials having the same standardized data, but different manufacturing dates for example, can be labeled with RFID tags storing different, vial-specific information. Thus, drug vials having different expiration dates and/or lot numbers, for example, but the same standardized data, can have different supplemental information stored in their respective RFID tags. Thus, the supplemental information for the first drug in the first drug vial 10 may be absent from the information related to the first drug encoded by the barcode 22.

The differentiator module 86, at block 94, compares: (i) a portion of the information about the first drug determined based on the barcode 22, to (ii) a portion of the data read from the RFID tags 24, 38 to generate a comparison result. For example, the comparison result can indicate whether the RFID tag 24 and/or the RFID tag 38 stores the portion of the information about the first drug that matches the corresponding information determined based on the barcode 22.

For example, the differentiator module 86 can determine that the drug name determined based on the barcode 22 matches the drug name determined based on the RFID tag 24. As another example, the differentiator module 86 can determine that the drug concentration determined based on the barcode 22 matches the drug concentration determined based on the RFID tag 24. As a result, the differentiator module 86 can determine, at block 96, that the information read from the RFID tag 24 memory 30 provided to the first RFID tag 24 corresponds to the first drug stored by the first drug vial 10 based on the comparison result.

At block 98, the content generation module 82 generates label content 60 for the first drug. According to some embodiments, the label content 60 includes a combination of: information based on the pattern data of the barcode 22 and information determined as a result of reading the memory 30 of the first RFID tag 24, which was determined to correspond to the first drug based on the comparison result. According to some embodiments, the label content 60 includes information determined as a result of reading the memory 30 of the first RFID tag 24, which was determined to correspond to the first drug based on the comparison result, but lacks information based on the pattern data of the barcode 22. The information determined as a result of reading the memory 30 of the first RFID tag 24 can optionally include a portion of the supplemental information.

The content generation module 82 can optionally include a source module 104 that controls, or interferes with the inclusion of information read from the RFID tag 24 in the label content 60 for the first drug. Certain parties are permitted to store information about the first drug in the memory 30 of the RFID tag 24 provided to the first drug vial 10. For example, sources of the first drug identified in the NDC can store drug information about the first drug in the memory 30 of the RFID tag 24. As a source of the drug registered with the FDA and assigned a source code included in the NDC, such registered sources (e.g., manufacturer, re-packager, distributor) are deemed to be a trustworthy or reliable source of drug information. In other words, the information stored in the memory 30 of the RFID tag 24 can be relied on, and the RFID tag 24 can be considered a source of truth when the RFID tag 24 is provided by a registered source. The source module 104 can be operable to control the inclusion of a portion of the information stored in the memory 30 of the RFID tag 24 from a reliable source in the label content 60.

Information stored in the memory 30 of the RFID tag 24 provided by an unreliable source, such as a third party that is not identified by the NDC for example, can optionally be used by the drug labeling apparatus 46, and/or may cause a warning to be issued by the drug labeling apparatus 46 (e.g., displayed on the display 54). The warning can notify a user of the drug labeling apparatus 46 that the drug information is from an unreliable source. The warning can optionally request the user to confirm the accuracy of the drug information, and not rely on the drug information without further investigation. Thus, the source module 104 can optionally cause the content generation module 82 to exclude the drug information from the unreliable source from the label content 60 until or unless user confirmation is received. According to some embodiments, the source module 104 can optionally cause the content generation module 82 to prevent the label content 60 from including the information from an unreliable source. However, the information from an unreliable source may still be of some value to the user, so such information can be displayed by the display 54 or otherwise conveyed virtually by the drug labeling apparatus 46 to the user.

For example, the drug labeling apparatus 46 can alert the user to a potential property of the first drug determined based on the information stored by the RFID tag 24 from an unreliable source. More specifically, the processing component 76 can determine the expiration date of the first drug based on information stored in the memory 30. Despite the information stored by the RFID tag 24 being supplied by an unreliable source, the display 54 can visually present the user with a notification that the drug is potentially expired based on this information. The notification can optionally indicate that the expiration date is provided based on information from an unreliable source. Such a notification can have a different visual appearance than a similar notification from a reliable source, which can at least alert the user to the possibility that the drug has expired. It is left to the user's discretion to confirm whether the drug is expired, as suggested by the information from the unreliable source.

According to some embodiments, the source module 104 can determine whether the information stored by the RFID tag 24 is supplied by a reliable source or an unreliable based on the information stored by the memory 30 of the RFID tag 24. For example, the unreliable sources are not permitted to include substantive drug information in the memory 30 of the RFID tag 24. Only a registered source can store substantive drug information in the memory 30. According to some embodiments, substantive drug data includes any information conveying a property or regulated information about the drug, such as information governed by a regulatory body such as the FDA, for example. Examples of substantive information can include at least one of: a name of the first drug, an expiration date of the first drug, and a lot number of the first drug. Any such information present within the memory 30 is indicative that the source of the information is a reliable source. Thus, source module 104 can identify the source as a reliable source in response to determining that the data read from the memory 30 provided to the first RFID tag 24 comprises substantive information about the first drug.

In contrast, the source module 104 can identify the source as an unreliable source of information in response to determining that the data read from the memory 30 of the RFID tag 24 lacks substantive information about the first drug. Instead, the information from an unreliable source can include a reference number, for example, that can be used by the drug labeling apparatus 46 to lookup information about the first drug from a memory external of, and remote from the RFID tag 24. For example, if the memory 30 of the RFID tag 24 lacks substantive information, but stores a reference number that must be used to lookup substantive information about the first drug from the formulary 84 or a remote database, for example, the source module 104 determines that the information stored in the memory 30 is from an unreliable source.

According to some embodiments, the source module 104 can identify the source of information stored in the memory 30 of the RFID tag 24 as being reliable or unreliable based on a source identifier that identifies the party responsible for the information. For example, if the data read from the memory 30 provided to the RFID tag 24 includes a NDC, which includes a segment that identifies the source, or another identifier that specifically identifies the source, the source module 104 can reference a database of sources to determine whether the source is included among a list of reliable sources or unreliable sources. The absence of the source from a listing of reliable sources can lead to the determination that the source is unreliable.

At block 100, a publishing module, such as the printer 68 for example, generates the drug label 62 that is to be applied to the drug delivery container 66 for the first drug. For example, the printer 68 can print or otherwise apply the label content 60 onto a surface of an adhesive-backed label stock. The label content can optionally include a computer readable code 102 (FIG. 3) that, when scanned by a compatible reading device, conveys the label content 60 to the reading device. According to some embodiments, the publishing module can include information from a reliable source, and/or information from an unreliable source that has been independently confirmed by the user in the label content applied to the drug label 62.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A drug labeling apparatus comprising:
an optical code reader comprising an optical imager that captures pattern data indicative of a pattern of symbols included in a first computer-readable code provided to a first drug vial, wherein the pattern data represents information related to a first drug stored by the first drug vial;
a RF code reader comprising an antenna, and a signal generator that generates an interrogation signal to be transmitted by the antenna to: (i) generate a field that encompasses a first RFID tag associated with the first drug vial and a second RFID tag associated with a second drug vial, and (ii) read data stored by non-transitory computer-readable media provided to the first RFID tag and the second RFID tag;
a differentiator module that:
compares: (i) a portion of the information about the first drug determined based on the first computer-readable code, and (ii) a portion of the data read from the non-transitory computer-readable media provided to the first RFID tag and the second RFID tag to generate a comparison result, and
determines that the data read from the non-transitory computer-readable medium provided to the first RFID tag corresponds to the first drug stored by the first drug vial based on the comparison result;
a content generation module that generates label content comprising information derived from the pattern data and information derived from the data read from the non-transitory computer-readable media provided to the first RFID tag; and
a publishing module that generates a drug label that is to be applied to a drug delivery container by applying the label content to an adhesive-backed label stock.

2. The drug labeling apparatus of claim 1, wherein the optical code reader comprises a barcode reader that reads the pattern data from a one-dimensional and/or two-dimensional barcode, and the barcode reader extracts a standardized code that is compliant with a standard approved by a regulatory body comprising a numeric, alphabetic, or alpha-numeric string from the pattern data to identify at least a name of the first drug.

3. The drug labeling apparatus of claim 2, wherein the standardized code comprises a National Drug Code comprising a string of numbers arranged in three segments, and the name of the first drug is encoded by one of the three segments.

4. The drug labeling apparatus of claim 2, wherein the RF code reader reads at least supplemental information, that is different from the information related to the first drug represented by the pattern data, from the non-transitory computer-readable medium provided to the first RFID tag.

5. The drug labeling apparatus of claim 4, wherein the supplemental information comprises at least one of: an expiration date of the first drug stored by the first drug vial, and a lot number assigned to the first drug stored by the first drug vial by a supplier of the first drug vial.

6. The drug labeling apparatus of claim 5, wherein the supplemental information is absent from the information related to the first drug encoded by the pattern data.

7. The drug labeling apparatus of claim 1 further comprising a source module that identifies a source of the data read from the non-transitory computer-readable medium provided to the first RFID tag, designates the source of the data to be a reliable source or an unreliable source and, in response to determining that the source of the data is the unreliable source, alerts a user of the drug labeling apparatus to a potential property of the drug.

8. The drug labeling apparatus of claim 7, wherein the source module identifies the source as the reliable source or the unreliable source based on a source identifier included in the data read from the non-transitory computer-readable medium provided to the first RFID tag.

9. The drug labeling apparatus of claim 7, wherein the source module identifies the source as the reliable source in response to determining that the data read from the non-transitory computer-readable medium provided to the first RFID tag comprises substantive information about the first drug, and identifies the source as the unreliable source in response to determining that the data read from the non-transitory computer-readable medium provided to the first RFID tag lacks substantive information about the first drug.

10. The drug labeling apparatus of claim 9, wherein the substantive information about the first drug comprises at least one of: a name of the first drug, an expiration date of the first drug, and a lot number of the first drug.

11. The drug labeling apparatus of claim 9, wherein the data read from the non-transitory computer-readable medium provided to the first RFID tag is determined to lack the substantive information about the first drug if the data read from the non-transitory computer-readable medium provided to the first RFID tag: (i) does not comprise a name of the first drug, an expiration date of the first drug, or a lot number of the first drug, and (ii) comprises a reference number that is used to lookup drug-specific information from a database.

12. The drug labeling apparatus of claim 7, wherein the source module interferes with operation of the publishing module to generate the drug label, and requires user confirmation of at least a portion of the label content before causing the publishing module to generate the drug label.

13. A method of producing a label for a drug delivery container comprising:
scanning, with an optical code reader comprising an optical imager, a barcode provided to a first drug vial to obtain information related to a first drug stored by the first drug vial;
reading, with a RF code reader, data stored by a non-transitory computer-readable medium provided to a first RFID tag associated with the first drug vial and data stored by a non-transitory computer-readable medium provided to a second RFID tag associated with a second drug vial;
comparing, with a differentiator module: (i) a portion of the information about the first drug determined based on the barcode, and (ii) a portion of the data read from the non-transitory computer-readable media provided to the first RFID tag and the second RFID tag to generate a comparison result;
determining, with the differentiator module, that the data read from the non-transitory computer-readable medium provided to the first RFID tag corresponds to the first drug stored by the first drug vial based on the comparison result;
generating, with a content generation module, label content comprising information derived from the barcode and information derived from the data read from the non-transitory computer-readable medium provided to the first RFID tag; and
producing, with a publishing module, a drug label that is to be applied to a drug delivery container for administering the first drug by applying the label content to an adhesive-backed label stock.

14. The method of claim 13, wherein scanning the barcode comprises:
extracting a standardized code that is compliant with a standard approved by a regulatory body comprising a numeric, alphabetic, or alpha-numeric string from the barcode; and
looking up an entry in a drug formulary using the standardized code to identify at least a name of the first drug.

15. The method of claim 14, wherein looking up the entry in the drug formulary comprises identifying a portion of a National Drug Code comprising a subset of numbers in the National Drug Code corresponding to the name of the first drug, and identifying the entry in the drug formulary by matching the subset of numbers to corresponding numbers included in the entry.

16. The method of claim 14, wherein reading the data stored by the non-transitory computer-readable medium provided to the first RFID tag comprises reading supplemental information, that is different from the information related to the first drug obtained from the barcode, from the non-transitory computer-readable medium provided to the first RFID tag.

17. The method of claim 16, wherein the supplemental information comprises at least one of: an expiration date of the first drug stored by the first drug vial, and a lot number assigned to the first drug stored by the first drug vial by a supplier of the first drug vial.

18. The method of claim 13 further comprising:
identifying, with a source module, a source of the data read from the non-transitory computer-readable medium provided to the first RFID tag; and
determining that the source of the data is a reliable source or an unreliable source and, in response to determining that the source of the data is the unreliable source, alerts a user of the drug labeling apparatus to a potential property of the drug.

19. The method of claim 18, wherein the source module identifies the source as the reliable source or the unreliable source based on a source identifier included in the data read from the non-transitory computer-readable medium provided to the first RFID tag.

20. The method of claim 18, wherein the source module interferes with operation of the publishing module to generate the drug label, and requires user confirmation of at least a portion of the label content before causing the publishing module to generate the drug label.
